(19) United States
(12) Patent Application Publication
(10) Pub. No.: US 2012/0276593 A1
(43) Pub. Date: Nov. 1, 2012

(54) USE OF CELLULASE AND GLUCOAMYLASE TO IMPROVE ETHANOL YIELDS FROM FERMENTATION

(75) Inventors: Mian Li, Santa Clara, CA (US); Colin Mitchinson, Half Moon Bay, CA (US)

(73) Assignee: Danisco US Inc., Palo Alto, CA (US)

(21) Appl. No.: 13/458,597

(22) Filed: Apr. 27, 2012

Related U.S. Application Data
(60) Provisional application No. 61/481,094, filed on Apr. 29, 2011.

(51) Int. Cl. C12P 19/20; C12N 9/16; C12N 9/34; C12P 19/22; C12P 7/10

(52) U.S. Cl. 435/95; 435/96; 435/165; 435/205; 435/196

(57) ABSTRACT

An improved saccharification process comprises the use of a glucoamylase and at least one cellulase. The improved saccharification process results in improved yields of fermentations products, such as ethanol. In one embodiment, the improved saccharification process results in an increased yield of up to 0.5% to 1% ethanol using commercially available cellulases. Also provided are improved simultaneous saccharification and fermentation (SSF) processes, and compositions comprising a liquefied starch slurry, a glucoamylase, and a cellulase.

### PRIORITY

The present application claims priority to U.S. Provisional Application Ser. No. 61/481,094, filed on Apr. 29, 2011, which is hereby incorporated by reference in their entirety.

### FIELD OF THE INVENTION

This relates to fermentation of starch and/or biomass, and in particular, processes for improving product yields from such fermentations, for example, the yield of ethanol. In particular, this relates to compositions and processes for producing ethanol from fermentations (including simultaneous saccharification and fermentation (SSF) processes) using glucoamylase and cellulase in combination to saccharify and/or ferment starch and/or biomass.

### BACKGROUND

The industrial fermentation of starch and/or biomass to make useful products, such as ethanol, continues to be an area of great interest. Among ethanol's many uses are applications in food and beverages, as well as an industrial chemical, a fuel additive, or a liquid fuel. Current economic, social, political, environmental, energy, and geologic concerns make fuel ethanol of particular interest. As a potential fuel source and because it is derived from renewable resources, ethanol may help reduce dependence on fossil fuel sources, reduce undesirable emissions, improve performance of gasoline engines, and decrease accumulation of carbon dioxide in the atmosphere.

While there has been interest in obtaining ethanol from the saccharification and fermentation of primarily cellulosic materials, the vast majority of ethanol is produced from the fermentation of starchy materials. A typical process of ethanol production from starch-containing raw materials comprises two sequential enzyme-catalyzed steps that result in the release of glucose from the starch prior to fermentation. The first step is liquefaction of the starch, catalyzed by alpha-amylases. Alpha-amylases (EC 3.2.1.1) are endohydrolases that randomly cleave internal α-1,4-D-glucosidic bonds. They are capable of degrading the starch slurry to shorter maltodextrins. As the alpha-amylases degrade the starch, the viscosity of the mixture decreases. Because liquefaction typically is conducted at high temperatures, thermostable alpha-amylases, such as an alpha-amylase from Bacillus sp., are preferentially used. Many new alpha-amylases have been developed in recent years to improve liquefaction, and to provide many interesting, novel, and useful enzymatic properties.

Enzymatic liquefaction can be a multi-step process. For example, after enzyme addition, the slurry is heated to a temperature between about 60-95° C., typically about 78-88° C. Subsequently, the slurry is heated, for example jet-cooked or otherwise, to a temperature typically between about 95-125° C., and then cooled to about 60-95° C. More enzyme(s) is (are) added, and the mash is held for another about 0.5-4 hours at the desired temperature, generally about 60-95° C. In some cases, cellulases are known to be added to a liquefaction tank to help reduce viscosity of the mash. Examples of commercial cellulase products which have been used for this purpose include various OPTIMASH™ by Danisco's Genencor Division, e.g. OPTIMASH™ BG, OPTIMASH™ TBG, OPTIMASH™ VR, and OPTIMASH™ XL.

Despite the reduction in viscosity and the cleavage of longer starch molecules to shorter maltodextrins during such liquefaction processes, these maltodextrins cannot be readily fermented by yeast to form alcohol. Thus, the second enzyme-catalyzed step, saccharification, may be required to further break down the maltodextrins. Glucoamylases and/or maltogenic alpha-amylases commonly are used to catalyze the hydrolysis of non-reducing ends of the maltodextrins formed after liquefaction to release glucose, maltose and isomaltose. Debranching enzymes, such as pullulanases, can also be used to aid saccharification. Saccharification generally is conducted under acidic conditions at elevated temperatures, e.g., about 60° C., pH 4.3.

While basic enzymatic starch liquefaction processes are well established, further improvements in commercial starch processing may be useful. In particular, cellulosic material remains after the milling of the raw material (e.g. grain, such as corn) and the gelatinization and liquefaction of the starch. This fibrous cellulosic material can entrap or bind some starch, thus reducing both theoretical and actual yields. A cellulase can be used during liquefaction to decrease the viscosity of the slurry. See, e.g., Öhgren et al., Process Biochemistry, Vol. 42, pp. 834-839, 2007. A cellulase also can be used in a SSF process for the pretreated lignocellulosic materials such as softwood pulp, or sugarcane bagasse. See, e.g., Kovács et al., Process Biochemistry, Vol. 44, pp. 1323-1329, 2009; and da Silva et al., Bioresource Technology, Vol. 101, pp. 7402-7409, 2010. Processes that can improve yields of fermentation products, such as ethanol, would represent an advance in the art, because even small reproducible improvements in yield, if attainable without additional energy input, are valuable when considered in view of the annual production of 12 billion gallons of ethanol in the U.S. alone.

### SUMMARY

Processes for saccharifying and fermenting starch-containing materials are provided. Product yields can be increased by saccharifying starchy plant materials (such as cereal grains) in the presence of a cellulase and a glucoamylase for fermentation stock. The processes involve adding a cellulase and a glucoamylase after liquefaction, e.g. preferably during saccharification and/or fermentation. The present processes differ from what has been known in the field—using a cellulase (1) during liquefaction to decrease the viscosity of the slurry (the cellulase is generally inactivated at the end of the high-temperature liquefaction step); and (2) in a SSF process for cellulose-rich materials having a low starch content. The enzymes may be added during simultaneous saccharification and fermentation (SSF), for example. Without limitation to any particular mode of action, the enzymes may hydrolyze some portion of the cellulosic material and/or help release starch molecules bound to or entrapped by cellulose fibers. Regardless of mechanism, the net effect of the inclusion of the enzymes is an increase in product yield, apparently due to the release/conversion of additional fermentable materials to produce additional glucose.

Distillers' dried grain with solubles (DDGS), which is a by-product or co-product of dry-grind ethanol facilities, generally contains about 20% or more total glucan, about 16% (dry weight basis) of which is from cellulose. (See Youngmi et al., Bioresource Technology, 99:5165-5176 (2008)). If fully converted to glucose, that cellulose could theoretically produce about an additional 0.1 gal of ethanol per bushel of corn. (Saville and Yacyshyn, “Effect of Cellulase Supplementation on Cookline Operation in A Dry Mill Ethanol Plant,” 27th Symposium on Biotechnology for Fuels and Chemicals, May 1-4, 2005, Denver, Colo.). For example, for ethanol fermentation, product yields can be increased by 0.4-0.5%. If applied industry-wide, such improvements would produce an additional 48-60 million gallons of ethanol in the U.S. annually.

Accordingly, in a first aspect, a method of saccharifying a starch-containing substrate to produce a fermentation stock is provided. The methods comprise (a) contacting a liquefied starch slurry (i.e., liquefact) that contains at least some cellulosic material with both a glucoamylase and a cellulase under conditions sufficient for enzyme activity, and (b) allowing time for the enzyme activity to occur, thereby producing a fermentation stock. Preferably the enzyme activity is sufficient to at least: (a) increase concentration of at least one fermentable sugar in the fermentation stock; (b) release at least one starch chain bound to or trapped by cellulose; or (c) to hydrolyze some portion of the cellulosic material present in the liquefied starch slurry.

In one embodiment, methods are provided for improving the yield of a fermentation product produced by fermenting a starch substrate. The methods generally comprise the steps of selecting a liquefied starch that contains at least some cellulosic material, contacting the liquefied starch with both a glucoamylase and a cellulase under conditions sufficient for enzyme activity, and subsequently fermenting the mixture to produce the fermentation product. In one presently preferred embodiment, the fermentation product is ethanol. The yield of fermentation product can be improved by about 0.1% to about 1.0% in various embodiments.

In a further embodiment methods are provided for simultaneously saccharifying and fermenting a liquefied cereal starch. Such methods comprise the steps of (1) contacting a liquefied starch slurry with a glucoamylase and a cellulase under conditions sufficient for enzyme activity and fermentation, in the present of an organism suitable for the fermentation, and (2) allowing the enzyme activity and fermentation to proceed. In one embodiment, the fermentation proceeds for at least 24 to about 72 hours. The fermentation may have an improved product yield relative to a control fermentation with no cellulase added. In one embodiment, the fermentation produces ethanol, and the ethanol yield is improved, for example by about 0.1 to about 1.0%.

In yet another aspect, compositions comprising a liquefied starch slurry, glucoamylase, and cellulase are provided. Such compositions are useful for preparing a feedstock for a fermentation for ethanol or other useful products.

### BRIEF DESCRIPTION OF THE DRAWINGS

FIG. 1 shows the effect of adding cellulase on ethanol yield during a 72 h fermentation of a 32% dry solids (DS) corn mash. The experiment used an SSF process. The glucoamylase was G-ZYME 480 (Danisco US Inc., Genencor Division) at 0.4 GAU/g corn. The cellulase was ACCELLERASE 1000 (Danisco US Inc., Genencor Division) added at 5 kg/metric ton dry corn. The control contained glucoamylase, but no cellulase was added. Ethanol, DP1, and DP2 concentrations were measured for the control and cellulase treatments. The y-axis shows the concentration (g/L); the x-axis reflects the hours of fermentation.

FIG. 2 is a bar chart showing the results of including 0, 5, 10, and 50 kg of cellulase enzyme per metric ton of dry solids (kg/MT DS) in the liquefact in the presence of a glucoamylase. The y-axis shows the amount of ethanol (g/L) at the indicated times.

FIGS. 3-4 show the results of one experiment adding glucoamylase (G-ZYME, (Danisco US Inc., Genencor Division) at 0.4 GAU/g corn) and cellulase (ACCELLERASE 1500 (Danisco US Inc., Genencor Division) 0.5-2 kg/MT DS) to corn mash fermentation.

FIG. 3 depicts the effect of glucoamylase and cellulase on ethanol yield. The chart shows the final amount of ethanol (% v/v) on the y-axis, relative to the amount of cellulase added (% w/w DS).

FIG. 4 shows the final concentration of glucose in the fermentation relative to the amount of cellulase added in the experiment depicted in FIG. 3. The chart shows the final glucose concentration (% w/v) on the y-axis, relative to the amount of cellulase added (% w/w DS).

### DETAILED DESCRIPTION

The processes provided herein comprise the use of a cellulase enzyme where saccharifying a starchy material after liquefaction. Inclusion of a cellulase in the saccharification or SSF of starchy material, such as cereal grains, can provide improved yields of fermentation products. The improved saccharification or SSF processes advantageously increases the concentration of glucose, releases one or more starch molecules bound to, associated with, or trapped by cellulosic material, or degrades at least some portion of the cellulose remaining after, e.g. dry milling and liquefaction. In one embodiment, the improved saccharification process results in an increased yield of ethanol using commercially available cellulases that are added with glucoamylases.

### 1. Definitions & Abbreviations

In accordance with this description, the following abbreviations and definitions apply. It should be noted that as used herein, the singular forms “a,” “an,” and “the” include plural referents unless the context clearly dictates otherwise. Thus, for example, reference to “an enzyme” includes a plurality of such enzymes, and reference to “the formulation” includes reference to one or more formulations and equivalents thereof known to those skilled in the art, and so forth. Also, as used herein, “comprising” and its cognates are used in their inclusive sense; that is, equivalent to the term “including” and its corresponding cognates.

Unless defined otherwise, all technical and scientific terms used herein have the same meaning as commonly understood by one of ordinary skill in the art. The following terms are provided below.

### 1.1. Definitions

The term “about” with respect to a numerical value or range indicates that the numerical value can be up to 10% greater or less than the stated value. In other embodiments, “about” indicates that a numerical value can be up to 5% greater or less than the stated value.

As used herein, “starch” refers to any material comprised of the complex polysaccharide carbohydrates of plants, comprised of amylose and amylopectin with the formula (C₆H₁₀O₅)ₓ, wherein X can be any positive integer. In particular, the term refers to any plant-based material including but not limited to grains, grasses, tubers, and roots. Preferably the starchy material is wheat, barley, corn, rye, oats, rice, sorghum or milo, brans, cassava, millet, potato, sweet potato, and tapioca. For purposes herein “sorghum” generally includes “grain sorghum”, also known as “milo”.

The term “slurry” refers to an aqueous mixture containing at least some insoluble solids. A slurry can also contain one or more soluble components. Milled grain, flour, or starch are frequently suspended in a water-based solution to form a slurry for testing amylases, or for liquefaction processes.

“Gelatinization” means solubilization of a starch molecule by cooking to form a viscous suspension.

The term “liquefaction” means a process by which starch is “liquefied” or converted to less viscous and shorter chain soluble dextrins. The process of liquefying involves gelatinization of starch simultaneously with, or followed by, the addition of at least an alpha-amylase. Thus, liquefaction is the stage in which gelatinized starch is enzymatically hydrolyzed, e.g. thereby reducing the chain length of the starch and concomitantly, the viscosity. As used herein “liquefact” refers to the liquefied starch slurry, i.e. the resultant hydrolyzed mixture. Such a liquefact is generally the starting material for a saccharification process in connection with a fermentation.

As used herein, “saccharification” refers to enzymatic conversion of starch to glucose. After liquefaction, a starch slurry is “saccharified” to convert the maltodextrins to fermentable sugars, e.g. glucose, maltose. Saccharification involves the use of enzymes, particularly glucoamylases, but also debranching enzymes are frequently used.

The term “degree of polymerization (DP)” refers to the number (n) of anhydroglucopyranose units in a given saccharide. Examples of DP1 are the monosaccharides, such as glucose and fructose. Examples of DP2 are the disaccharides, such as maltose and sucrose. A DP>3 denotes polymers with a degree of polymerization of greater than 3. DP can be used a measure of the relative degree of breakdown of starch (high DP) to sugars (low DP). The term “DE,” or “dextrose equivalent,” is defined as the percentage of reducing sugar as a fraction of total carbohydrate.

“Simultaneous saccharification and fermentation” (SSF) refers to a specific type of fermentation process wherein a step of saccharifying a raw material (e.g. a whole grain or other biomass comprising a starch and a cellulosic material) and a fermentation step are combined into a single process that is conducted together.

“Amylase” means an enzyme that is, among other things, capable of catalyzing the degradation of starch, amylose, amylopectin, and the like. Generally, amylases include (a) endo-cleaving enzyme activity (e.g. as found in α-amylases (EC 3.2.1.1; α-D-(1→4)-glucan glucanohydrolase)) cleaving α-D-(1→4) O-glycosidic linkages in a polysaccharide containing three or more α-D-(1→4) linked glucose units, and (b) the exo-cleaving amylolytic activity that sequentially cleaves the substrate molecule from the non-reducing end. Examples of the latter are found in β-amylases (EC 3.2.1.2), which produce β-maltose. β-Amylases, α-glucosidases (EC 3.2.1.20; α-D-glucoside glucohydrolase), glucoamylase (EC 3.2.1.3; α-D-(1→4)-glucan glucohydrolase), and product-specific amylases can produce malto-oligosaccharides of a specific length from their respective substrates.

“Alpha-amylase” (e.g., E.C. 3.2.1.1) generally refers to enzymes that catalyze the hydrolysis of alpha-1,4-glucosidic linkages. These enzymes effect the hydrolysis of 1,4-α-D-glucosidic linkages in polysaccharides containing 1,4-α-linked D-glucose units. The alpha-amylases release the reducing groups in the α-configuration. For the purpose of the present disclosure, “alpha-amylase” particularly includes those alpha amylase enzymes having relatively high thermostability, i.e., with sustained activity at high temperatures. For example, alpha-amylases are useful for liquefying starch at temperatures above 80° C.

“Activity” with respect to enzymes means catalytic activity and encompasses any acceptable measure of enzyme activity, such as the rate of activity, the amount of activity, or the specific activity. As used herein, “specific activity” means an enzyme unit defined as the number of moles of substrate converted to product by an enzyme preparation per unit time under specific conditions. Specific activity is expressed as units (U)/mg of protein.

“Alpha-amylase unit” (AAU) refers to alpha-amylase activity measured according to the method disclosed in U.S. Pat. No. 5,958,739. In brief, the assay uses p-nitrophenyl maltoheptoside (PNP-G7) as the substrate with the non-reducing terminal sugar chemically blocked. PNP-G7 can be cleaved by an endo-amylase, for example alpha-amylase. Following the cleavage, an alpha-glucosidase and a glucoamylase digest the substrate to liberate free PNP molecules, which display a yellow color and can be measured by visible spectrophotometry at 410 nm. The rate of PNP release is proportional to alpha-amylase activity. The AAU of a given sample is calculated against a standard control. One unit of AAU refers to the amount of enzyme required to hydrolyze 10 mg of starch per minute under specified conditions.

“Glucoamylases” are a type of exo-acting amylase that release glucosyl residues from the non-reducing ends of amylose and amylopectin molecules. Glucoamylases also catalyze the hydrolysis of α-1,6 and α-1,3 linkages, although at much slower rate than α-1,4 linkages. Glucoamylase activity can be expressed in “glucoamylase units” (GAU).

“Cellulose” as used herein is a generic term that includes cellulose, hemi-cellulose, lignins, related beta-D-glucans, and the like.

As used herein, “cellulases” refer to all enzymes that hydrolyzes cellulose, i.e., any of its components, e.g., 1,4-beta-D-glycosidic linkages in cellulose, hemi-cellulose, lignin and/or related beta-D-glucans such as those found in cereals. Thus, encompassed within “cellulase” are at least all those enzymes classified as E.C. 3.2.1.4 (cellulase/endocellulases), E.C. 3.2.1.91 (exocellulases), and E.C. 3.2.1.21 (cellobiases). Examples of endocellulases include endo-1,4-beta-glucanase, carboxymethyl cellulase (CMCase), endo-1,4-beta-D-glucanase, beta-1,4-glucanase, beta-1,4-endoglucan hydrolase, and celludextrinase. Examples of exocellulases include cellobiohydrases that work from the reducing ends and those that work on the non-reducing ends of cellulose molecules. Beta glucosidases are another name for cellobiases. In certain embodiments herein, cellulase refers preferentially to one or more of endocellulase, exocellulase, hemicellulase and beta-glucosidase, or any combinations thereof. Commercial preparations of cellulase compositions are suitable for use herein, including for example, products of Danisco's Genencor Division, such as ACCELLERASE 1000 and ACCELLERASE 1500, which contain exo- and endo-glucanases, a hemicellulase, and a beta glucosidase.

The terms “protein” and “polypeptide” are used interchangeably herein.

The term “derived” encompasses the terms “originated from,” “obtained” or “obtainable from,” and “isolated from.”

“Fermentation” is the enzymatic and/or anaerobic breakdown of organic substances by microorganisms to produce simpler organic compounds. While fermentation occurs under anaerobic conditions it is not intended that the term be solely limited to strict anaerobic conditions, as fermentation also occurs in the presence of oxygen at various levels. Fermentation encompasses at least any fermentative bioconversion of a starch substrate containing granular starch to an end product (for example, in a vessel or reactor).

The term “contacting” refers to the placing of the respective enzyme(s) in a reactor, vessel, or the like, such that the enzyme can come into sufficiently close proximity to the respective substrate so as to enable the enzyme(s) to convert the substrate to the end product. Those skilled in the art will recognize that mixing an enzyme (e.g. in solution) with one or more respective substrates, whether in a relatively pure or crude form, can effect contacting.

As used herein the term “dry solids content (ds)” refers to the total solids of a mixture (e.g. a slurry) on a dry weight basis. Dry solids content and dry weight basis are usually expressed, for example, as the weight of the subject material as a percentage of the weight of the total dry material.

The term “residual starch” refers to the amount of starch present in grain by-products after fermentation. Typically, the amount of residual starch present in 100 grams of DDGS may be one of the parameters to evaluate the efficiency of starch utilization in a fermentation process, such as an ethanol production process.

As used herein, “a recycling step” refers to the recycling of mash components, which may include residual starch, enzymes and/or microorganisms to ferment substrates comprising starch.

The term “mash” refers to a mixture of a fermentable carbon source (carbohydrate) in water used to produce a fermented product, such as an alcohol. In some embodiments, the term “beer” and “mash” are used interchangeability.

The term “stillage” means a mixture of non-fermented solids and water, which is the residue after removal of alcohol from a fermented mash.

The terms “distillers' dried grain” (DDG) and “distillers' dried grain with solubles” (DDGS) refer to a useful by-product of grain fermentation.

“Microorganism” as used herein includes any bacterium, yeast, or fungus species.

As used herein, “ethanologenic microorganism” refers to a microorganism with the ability to convert a sugar or oligosaccharide to ethanol. The ethanologenic microorganisms are ethanologenic by virtue of their ability to express one or more enzymes that individually or together convert sugar to ethanol.

As used herein, “ethanol producer” or “ethanol producing microorganism” refers to any organism or cell that is capable of producing ethanol from a hexose or pentose. Generally, ethanol-producing cells contain an alcohol dehydrogenase and a pyruvate decarboxylase. Examples of ethanol producing microorganisms include fungal microorganisms such as yeast. The typical yeast used in ethanol production includes species and strains of Saccharomyces, e.g., S. cerevisiae.

The term “heterologous” with reference to a polynucleotide or protein refers to a polynucleotide or protein that does not naturally occur in a host cell. In some embodiments, the protein is a commercially important industrial protein. It is intended that the term encompass proteins that are encoded by naturally occurring genes, mutated genes, and/or synthetic genes.

The term “endogenous” with reference to a polynucleotide or protein refers to a polynucleotide or protein that occurs naturally in the host cell.

The terms “recovered,” “isolated,” and “separated” as used herein refer to a compound, protein, cell, nucleic acid or amino acid that is removed from at least one component with which it is naturally associated.

The term “yield” with reference to the ethanol yield refers to the production of a compound, e.g., ethanol, from a certain amount of a starting material. “Yield” may be expressed as the product formed over a particular amount of time from the starting material. In one embodiment, the ethanol yield is calculated as “gal UD/bushel corn,” reflecting gallon of undenatured ethanol produced per bushel of corn. A bushel of corn weighs about 56 pounds.

“ATCC” refers to American Type Culture Collection located at Manassas, Va. 20108 (ATCC).

### 1.2. Abbreviations

The following abbreviations apply unless indicated otherwise:

AA alpha-amylase
AAU alpha-amylase unit
CMC carboxymethylcellulose
DDG distillers' dried grains
DDGS distillers' dried grain with solubles

### DE Dextrose Equivalent

DNA deoxyribonucleic acid
DPn degree of polymerization with n subunits
DS, ds dry solids
EC enzyme commission for enzyme classification
g gram
gal gallon
GAU glucoamylase activity unit
h hour
kg kilogram
MT metric ton
nm nanometer
pI isoelectric point
PNP-G₇ p-nitrophenyl maltoheptoside
ppm parts per million
rpm revolutions per minute
SSF simultaneous saccharification and fermentation
w/v weight/volume
w/w weight/weight
UD undenatured
μL microliter

### 2. Ethanol Production

In one of its aspects, the improved saccharification processes provided herein are particularly useful in the production of ethanol. In general, alcohol (ethanol) production from starch-containing materials can generally be separated into four steps: milling, liquefaction, saccharification, and fermentation.

### 2.1. Raw Materials

In the starch processing of the present disclosure, particularly in the ethanol processes of the present disclosure, the starting raw material is preferably a material that comprises both a substantial source of starch and a source of at least some cellulosic material. Typically, the raw material (for example, corn kernels) contains less than 5% cellulosic material (based on dry mass). See, e.g., Kim et al., Bioresour. Technol. Vol. 99, pp. 5177-5192 (2008). In one embodiment, the starch and the cellulose are closely associated in the natural state. In one typical application, the source of starch for use herein is a whole grain or at least mainly whole grain. The raw material may be chosen from a wide variety of starch-containing crops including corn, potato, cassava, sorghum or milo, wheat, barley, rye, oats, and the like. In one embodiment, the starch-containing raw material is cereal grain. In certain preferred embodiments, the starch-containing raw material can be whole grain selected from the group consisting of corn, wheat, and barley, or any combination thereof.

### 2.2. Milling

The grain is milled in order to open up the structure and allow for further processing. Three commonly used processes are wet willing, dry milling, and various fractionation schemes. In dry milling, the whole kernel is milled and used in the subsequent steps of the process. On the other hand, wet milling gives a very good separation of germ and meal (starch granules and protein), so that it is usually applied, with a few exceptions, at locations where there is a parallel production of syrups. Different fractionation processes, such as variations of the wet or dry milling processes, result in greater or lesser separation of the grain components. Dry milling is the most frequent milling method for ethanol fermentations. It is contemplated that for use herein, a highly purified starch is not required, and preferably, at least some residue of cellulose will remain associated with the starch. Accordingly, dry milling is well-suited for use with the disclosed processes.

### 2.3. Gelatinization and Liquefaction

In some embodiments, the starch substrate prepared as described above is slurried with water. The starch slurry may contain starch as a weight percent of dry solids of about 10-55%, about 20-45%, about 30-45%, about 30-40%, or about 30-35%. In one presently preferred embodiment the ds content can be between about 20% and about 35%. The pH of the slurry may be adjusted, for example with NaOH or HCl, as is useful or needed, for example to maximize enzyme stability and/or activity. It is sometimes beneficial to adjust the pH so as to improve or optimize alpha-amylase stability and activity.

For liquefaction herein, any conventional liquefaction processes is suitable, as are other less conventional liquefaction methods. Alpha-amylase can be used at any effective amount to accomplish the goals of liquefaction. Doses higher than conventionally used may be used herewith. Also contemplated for use herewith are varied times and/or temperature of liquefaction, provided they are effective to accomplish the viscosity reduction and starch breakdown required. While any alpha-amylase suited for liquefaction may be used, representative alpha-amylases contemplated for use herein include GC 358 and SPEZYME® XTRA (Danisco US Inc., Genencor Division), and LIQUOZYME® SC and LIQUOZYME® SC DS (Novozymes A/S, Denmark). Alternatively, alpha-amylase products including but not limited to SPEZYME® FRED, SPEZYME® HPA, Maxalig™ ONE (Danisco US Inc., Genencor Division) and FUELZYME® LF (Verenium Corp.) can be used for liquefaction. Combinations or blends of any of the foregoing enzyme products may also be used.

Conventional high temperature treatment, such as jet-cooking, typically performed at a temperature between about 100-125° C., are also contemplated for use herein, as are liquefaction processes that omit high temperature steps. The presence of residual alpha-amylase in the liquefact is not objectionable for the improved saccharification methods provided herein.

### 2.4. Improved Saccharification Methods

Following liquefaction, the mash or liquefact is further hydrolyzed through saccharification to produce low molecular sugars (DP1-DP2) that are can be readily fermented. In some embodiments, a pre-saccharification step of 1-4 hours may be included between the liquefaction step and the saccharification step. During saccharification, the hydrolysis is generally accomplished enzymatically by the presence of a glucoamylase. Typically, an alpha-glucosidase and/or an acid alpha-amylase may also be supplemented in addition of the glucoamylase.

In the improved saccharification methods, cellulase is added along with glucoamylase for example as described below.

In one aspect, improved methods of saccharifying a starch-containing substrate, such as a cereal grain or other starchy crop are provided. The methods are useful for preparing a fermentation feedstock for example. The methods comprise identifying a liquefied starch slurry (liquefact) that contains at least some cellulosic material; contacting the liquefact with both a glucoamylase and a cellulase under conditions sufficient for enzyme activity; and allowing time for the enzyme activity to occur. A fermentation feedstock is produced by the method and is useful for any type of fermentation whether to produce an industrial chemical, a pharmaceutical, or even ethanol or other biofuel.

In one embodiment, the enzyme activity, particularly the activity of the cellulase is sufficient to at least: (a) increase concentration of at least one fermentable sugar in the fermentation stock; (b) release at least one starch chain bound to or trapped by cellulose; or (c) to hydrolyze some portion of the cellulosic material. Experiments can be conducted wherein any of (a), (b), or (c) above may be measured relative to a control liquefact not contacted with or treated with cellulase in the saccharification process.

As the skilled artisan will appreciate “cellulase” activity is a complex group of enzyme activities and not a single protein or polypeptide with the ability to catalyze hydrolysis of a multitude of glucan linkages. In various embodiments, cellulase thus comprises any enzyme commonly considered or referred to as a cellulase, including any one or more of exoglucanase, endoglucanase, hemi-cellulase, beta-glucosidase, or xylanase activities, or any combinations thereof. The cellulase comprises at least exoglucanase, endoglucanase, hemi-cellulase, and beta-glucosidase activities. Some examples of commercial cellulases contemplated for use herein are provided in Section 3.3 below.

The cellulase can be added at any useful dose. The skilled artisan will appreciate that excessive application of any enzyme could have negative consequences, for example on fermentation simultaneous with or subsequent to a saccharification treatment. In various embodiments, the cellulase is added at a dose of between about 0.05 to about 50 kg/metric ton of dry solids in the liquefact, between about 0.075 to about 25 kg/metric ton, between about 0.1 to about 12.5 kg/metric ton, between about 0.3 to about 6 kg/metric ton, or between about 0.5 to about 1 kg/metric ton dry solids in the liquefact. In one presently preferred embodiment, about 5 kg cellulase/metric ton dry solids in the liquefact can be used.

Alternatively, the cellulase can be dosed relative to the glucoamylase added. In various embodiments, the cellulase/glucoamylase ratio can be between about 0.00011 to about 0.14 g/GAU, between about 0.00017 to about 0.07 g/GAU, between about 0.00022 to about 0.04 g/GAU, between about 0.00068 to about 0.016 g/GAU, or between about 0.0011 to about 0.0028 g/GAU.

It can be noted that while any saccharification of a starchy liquefact could potentially include a cellulase without any adverse consequence on the saccharification, from at least an economic perspective, it is useful to identify a liquefact that would benefit from the inclusion of cellulase. Thus, for example a liquefact known to contain or likely to contain cellulosic material would be a liquefact arising from dry milled starch sources, such as grain, particularly corn.

The contacting step can be sequential, with either the glucoamylase or cellulase being added first. The contacting step can also be simultaneous, with the enzymes being added at or about the same time. To avoid problems, it is preferred to select enzymes that share conditions for activity—e.g. overlapping pH, temperature, ionic strength, salt and/or other requirements such that conditions are more readily set for the enzymes to be active.

Saccharification can be further improved in one embodiment by contacting the liquefact with one or more additional enzymes selected from the group consisting of a debranching enzyme, a pectinase, a beta amylase, and a phytase. Such enzymes are useful for breaking down plant wall and other cellular material that remains after milling, and which is not affected by the liquefaction process. Additional digestion of such material may aid in the production of glucose or its equivalents, either directly (through release of reducing sugars) or indirectly (e.g. through releasing starch molecules trapped or bound by other materials, e.g. cellulose).

In another aspect, methods of improving yield of a fermentation product produced from fermenting a starch substrate are provided. As with the previous methods, the step of adding cellulase during saccharification of a starch-containing material after its liquefaction is involved. The methods generally comprise the steps of selecting a liquefied starch that contains at least some cellulosic material, contacting the liquefied starch with both a glucoamylase and a cellulase under conditions sufficient for enzyme activity, and subsequently fermenting the mixture to produce the fermentation product.

The selection step is generally similar to the identifying step above in that the advantages of the improved methods will accrue more readily to a properly selected liquefact—i.e. one that preferably has at least some cellulosic material present. In one embodiment, the fermentation product is ethanol. Preferably, the yield improvement is at least about 0.1% to about 1.0%. For ethanol production improvements of at least 0.3% to about 0.5% are achievable in practice.

The cellulase generally contains one or more of exoglucanase, endoglucanase, hemi-cellulase, beta-glucosidase, or xylanase activities, or any combination thereof. In one embodiment, the cellulase comprises at least exoglucanase, endoglucanase, hemi-cellulase, and beta-glucosidase activities.

Doses of cellulase are similar to those discussed above. Accordingly, cellulases may be added at between about 0.05 to about 50 kg/metric ton of dry solids in the liquefact. Generally, commercial cellulases such as ACCELLERASE products (Danisco US Inc., Genencor Division) can be dosed at about 5 kg/metric ton dry solids.

The contacting and fermenting steps can take any amount of time that is useful for yield and other considerations. Preferably, these two steps take about 24 to 72 hours total, i.e., collectively. However, the saccharification step alone can last several days if required. In one embodiment, the starch being saccharified is from corn, wheat, barley, sorghum or milo, rye, potatoes, or any combination thereof. More preferably, the starch can be from corn, e.g. a corn mash.

The methods may further comprise a step of contacting the liquefact with one or more additional enzymes selected from the group consisting of a debranching enzyme, a pectinase, a beta amylase, and a phytase, as discussed above.

As the skilled artisan will appreciate, full saccharification may take up to about 72 hours. In some embodiments, which may be presently preferred for use herein, the saccharification step and fermentation step are combined into a single step, referred to as simultaneous saccharification and fermentation or SSF.

In connection with SSF, another aspect provides improved methods of simultaneously saccharifying and fermenting a liquefied starch comprising contacting a liquefact with a glucoamylase and a cellulase under conditions sufficient for enzyme activity and fermentation, in the present of an organism suitable for the fermentation, and allowing the enzyme activity and fermentation to proceed for at least 24 to about 72 hours; wherein the fermentation has an improved product yield relative to a control fermentation with no cellulase added.

As with other aspects disclosed herein, the fermentation in one embodiment produces ethanol, and the ethanol yield is improved. Likewise, the cellulase comprises any one or more of exoglucanase, endoglucanase, hemi-cellulase, beta-glucosidase, or xylanase activities. Certain presently preferred cellulases comprise at least exoglucanse, endoglucanase, hemi-cellulase, and beta-glucosidase activities.

Dosing of cellulase can be any amount that is useful, with cellulase added at between about 0.05 to about 50 kg/metric ton of dry solids in the liquefact, between about 0.1 to about 25 kg/metric ton, between about 1 to about 10 kg/metric ton, or at about 2.5-7.5 kg/metric ton dry solids in the liquefact. In one presently preferred embodiment, about 5 kg cellulase/metric ton dry solids in the liquefact are used.

In various embodiments the yield is improved by about 0.1 to about 1.0%, or by about 0.3 to about 0.6%.

In one embodiment the contacting and fermenting steps combined take about 24 to 72 hours. The starch is from corn, wheat, barley, sorghum or milo, rye, potatoes, or combinations thereof in various embodiments. An exemplary starch is corn, particularly in connection with ethanol fermentation.

Additional enzymes such as debranching enzymes, pectinase, beta amylase, and phytase can be included optionally in the improved methods.

In another aspect of improved saccharification, compositions comprising a liquefact of a starch, a glucoamylase, and a cellulase are provided. The compositions are useful for preparing a feedstock for fermentation. The compositions can be stored, for example at temperatures below those which are useful for enzyme activity, and can be later warmed and held for further saccharification in accordance with the foregoing. Preferably, the compositions comprise cellulase at between about 0.05 to about 50 kg/metric ton of dry solids in the liquefact. The starch is generally from corn, wheat, barley, sorghum or milo, rye, potatoes, or any combination thereof, but corn is presently preferred, particularly for ethanol fermentation. The composition can also include one or more additional enzymes such as debranching enzymes, pectinase, beta amylase, and/or phytase.

### 2.5. Fermentation

The organism used in fermentations will depend on the desired end product. Typically, if ethanol is the desired end product, yeast will be used as the fermenting organism. In some embodiments, the ethanol-producing microorganism is a yeast, and specifically Saccharomyces, such as strains of S. cerevisiae (U.S. Pat. No. 4,316,956). A variety of S. cerevisiae are commercially available and these include but are not limited to Ethanol Red™ (Fermentis), THERMOSACC® and Superstart™ (Lallemand Ethanol Technology), FALI (Fleischmann's Yeast), FERMIOL® (DSM Specialties), Bio-Ferm® XR (NACB), and Angel alcohol yeast (Angel Yeast Company, China). The amount of starter yeast employed in the methods is an amount effective to produce a commercially significant amount of ethanol in a suitable amount of time, (e.g. to produce at least 10% ethanol from a substrate having between 25-40% ds in less than 72 hours). Yeast cells can be supplied in amounts of about 10⁴ to 10¹², and typically from about 10⁷ to 10¹⁰ viable yeast count per ml of fermentation broth. The fermentation will include in addition to a fermenting microorganisms (e.g., yeast), nutrients, optionally additional enzymes, including but not limited to phytases. The use of yeast in fermentation is well known. See, e.g., THE ALCOHOL TEXTBOOK, K. A. JACQUES ET AL., EDS. 2003, NOTTINGHAM UNIVERSITY PRESS, UK.

The improved method as described herein may result in an improved ethanol yield. The improved ethanol yield is about 0.1 to about 1.0% greater than that of an ethanol production process not featuring the glucoamylase and the added cellulase. The ethanol yield may be expressed as “gal UD/bushel corn,” reflecting gallon of undenatured ethanol produced per bushel corn. Modern technologies typically allow for an ethanol yield in the range of about 2.5 to about 2.8 gal UD/bushel corn. See Bothast & Schlicher, “Biotechnological Processes for Conversion of Corn into Ethanol,” Appl. Microbiol. Biotechnol., 67: 19-25 (2005). The improved ethanol production efficiency may attribute to more efficient starch utilization in the starch processing as described herein. At the end of ethanol production, the residual starch present in 100 gram of grain by-products is at least about 10%, about 20%, or about 30% lower than that of an ethanol production process having starch liquefied at a temperature of about 85° C. and at a alpha-amylase dosage required to reach a DE value of at least about 10 within 90 minutes.

In further embodiments, by use of appropriate fermenting microorganisms as known in the art, the fermentation end product may include without limitation glycerol, 1,3-propanediol, gluconate, 2-keto-D-gluconate, 2,5-diketo-D-gluconate, 2-keto-L-gulonic acid, succinic acid, lactic acid, amino acids and derivatives thereof. More specifically, when lactic acid is the desired end product, a Lactobacillus sp. (L. casei) may be used; when glycerol or 1,3-propanediol are the desired end products, E. coli may be used; and when 2-keto-D-gluconate, 2,5-diketo-D-gluconate, and 2-keto-L-gulonic acid are the desired end products, Pantoea citrea may be used as the fermenting microorganism. The above enumerated list are only examples and one skilled in the art will be aware of a number of fermenting microorganisms that may be appropriately used to obtain a desired end product.

A suitable variation on the standard batch system is the “fed-batch fermentation” system. In this variation of a typical batch system, the substrate is added in increments as the fermentation progresses. Fed-batch systems are useful when catabolite repression likely inhibits the metabolism of the cells and where it is desirable to have limited amounts of substrate in the medium. Measurement of the actual substrate concentration in fed-batch systems is difficult and is therefore estimated on the basis of the changes of measurable factors, such as pH, dissolved oxygen and the partial pressure of waste gases, such as CO₂. Batch and fed-batch fermentations are common and well known in the art.

Continuous fermentation is an open system where a defined fermentation medium is added continuously to a bioreactor, and an equal amount of conditioned medium is removed simultaneously for processing. Continuous fermentation generally maintains the cultures at a constant high density, where cells are primarily in log phase growth. Continuous fermentation allows for the modulation of one or more factors that affect cell growth and/or product concentration. For example, in one embodiment, a limiting nutrient, such as the carbon source or nitrogen source, is maintained at a fixed rate and all other parameters are allowed to moderate. In other systems, a number of factors affecting growth can be altered continuously while the cell concentration, measured by media turbidity, is kept constant. Continuous systems strive to maintain steady state growth conditions. Thus, cell loss due to medium being removed should be balanced against the cell growth rate in the fermentation. Methods of modulating nutrients and growth factors for continuous fermentation processes, as well as techniques for maximizing the rate of product formation, are well known in the art of industrial microbiology.

### 2.6. Distillation

Optionally, following fermentation, ethanol may be extracted by, for example, distillation and optionally followed by one or more process steps. In some embodiments, the yield of ethanol produced by the present methods will be at least about 8%, at least about 10%, at least about 12%, at least about 14%, at least about 15%, at least about 16%, at least about 17%, at least about 18%, and at least about 23% v/v. The ethanol obtained according to the process of the present disclosure may be used as, for example, fuel ethanol, drinking ethanol, i.e., potable neutral spirits, or industrial ethanol.

### 2.7. By-Products

Grain by-products from the fermentation typically are used for animal feed in either a liquid form or a dried form. If the starch is wet milled, non-starch by-products include crude protein, oil, and fiber, e.g., corn gluten meal. If the starch is dry-milled, the by-products may include animal feed co-products, such as distillers' dried grains (DDG) and distillers' dried grain plus solubles (DDGS). When the grain is dry milled and mixed in a slurry before liquefaction and saccharification, however, no grain is left as a by-product.

### 3. Enzymes Involved in Ethanol Production from Starch

In terms of the improved saccharification processes disclosed herein, such methods are useful in conjunction with various enzymes, some of which are known for use in preparing starch material for fermentations, such as to ethanol.

In some embodiments, additional enzymes can be included in either a liquefaction step of in the improved saccharification processes or SSF processes disclosed herein. Examples of such enzymes include alpha amylases which may be added in the liquefaction step, and may also be added in the saccharification step or carried over from the liquefaction step. Other examples include the cellulases and phytases, which can also be added in both the liquefaction and saccharification steps as discussed above. Other enzymes which can be added at one or more points during starch breakdown include glucoamylases, pectinases, debranching enzymes, and beta-amylases. Some additional aspects and/or sources of these enzymes are discussed below.

### 3.1. Alpha-Amylases

Any alpha-amylases useful in liquefaction and/or saccharification of starch substrates are contemplated for use herein. Particularly useful are those displaying relatively high thermostability and thus capable of liquefying starch at a temperature above 80° C. Alpha-amylases suitable for the liquefaction process may be from fungal or bacterial origin, particularly alpha-amylases isolated from thermophilic bacteria, such as Bacillus species. These Bacillus alpha-amylases are commonly referred to as “Termamyl-like alpha-amylases.” Well-known Termamyl-like alpha-amylases include those from B. licheniformis, B. amyloliquefaciens, and Geobacillus stearothermophilus (previously known as Bacillus stearothermophilus). Other Termamyl-like alpha-amylases include those derived from Bacillus sp. NCIB 12289, NCIB 12512, NCIB 12513, and DSM 9375, which are disclosed in WO 95/26397. Contemplated alpha-amylases may also derive from Aspergillus species, e.g., A. oryzae and A. niger alpha-amylases. In addition, commercially available alpha-amylases and products containing alpha-amylases include TERMAMYL™ SC, FUNGAMYL™, LIQUOZYME® SC and SAN™ SUPER (Novozymes A/S, Denmark), and SPEZYME® XTRA, GC 358, SPEZYME® FRED, SPEZYME® FRED-L, and SPEZYME® HPA (Danisco US Inc., Genencor Division).

Alpha-amylases useful herein include wild-type (or parent) enzymes, as well as variants of the parent enzyme. Such variants may have about 80% to about 99% sequence identity to a Termamyl-like alpha-amylase or other wild-type amylase such as the Bacillus licheniformis alpha-amylase (disclosed in US 2009/0238923, filed Nov. 3, 2008) or Geobacillus stearothermophilus alpha-amylase (disclosed in US 2009/0252828, filed Nov. 3, 2008). Amylase variants disclosed in WO 96/23874, WO 97/41213, and WO 99/19467 are also contemplated for use herein, including the Geobacillus stearothermophilus alpha-amylase variant having the mutations Δ(181-182)+N193F compared to the wild-type alpha-amylase disclosed in WO 99/19467.

In some embodiments, a variant alpha-amylase may display one or more altered properties compared to those of the parent enzyme. The altered properties may advantageously enable the variant alpha-amylase to perform effectively in liquefaction. Similarly, the altered properties may result in improved performance of the variant compared to its parent. These properties may include substrate specificity, substrate binding, substrate cleavage pattern, thermal stability, pH/activity profile, pH/stability profile, stability towards oxidation, stability at lower levels of calcium ion (Ca²⁺), and/or specific activity. Representative alpha-amylase variants include those disclosed in US 2008/0220476, published Sep. 11, 2008; US 2008/0160573, published Jul. 3, 2008; US 2008/0153733, published Jun. 26, 2008; and US 2008/0083406, published Apr. 10, 2008. Blends of two or more alpha-amylases, each of which may have different properties are also contemplated for use herein.

Alpha-amylase activity may be determined according to the method disclosed in U.S. Pat. No. 5,958,739, with minor modifications. In brief, the assay uses p-nitrophenyl maltoheptoside (PNP-G₇) as the substrate with the non-reducing terminal sugar chemically blocked. PNP-G₇ can be cleaved by an endo-amylase, for example alpha-amylase. Following the cleavage, an alpha-glucosidase and a glucoamylase digest the substrate to liberate free PNP molecules, which display a yellow color and can be measured by visible spectrophotometry at 410 nm. The rate of PNP release is proportional to alpha-amylase activity. The alpha-amylase activity of a sample is calculated against a standard control.

Variant or mutant alpha-amylases can also be made by the skilled artisan for use herein, beginning for example with any known wild-type sequence. Many methods for making such variants, e.g. by introducing mutations into known genes, are well known in the art. The DNA sequence encoding a parent α-amylase may be isolated from any cell or microorganism producing the α-amylase in question, using various methods well known in the art.

### 3.2. Glucoamylases

Another enzyme contemplated for use in the starch processing, especially during saccharification, is a glucoamylase (EC 3.2.1.3). Glucoamylases are commonly derived from a microorganism or a plant. For example, glucoamylases can be of fungal or bacterial origin.

Exemplary fungal glucoamylases are Aspergillus glucoamylases, in particular A. niger G1 or G2 glucoamylase (Boel et al. (1984), EMBO J. 3(5): 1097-1102), or variants thereof, such as disclosed in WO 92/00381 and WO 00/04136; A. awamori glucoamylase (WO 84/02921); A. oryzae glucoamylase (Agric. Biol. Chem. (1991), 55(4): 941-949), or variants or fragments thereof. Other contemplated Aspergillus glucoamylase variants include variants with enhanced thermal stability: G137A and G139A (Chen et al. (1996), Prot. Eng. 9: 499-505); D257E and D293E/Q (Chen et al. (1995), Prot. Eng. 8: 575-582); N182 (Chen et al. (1994), Biochem. J. 301: 275-281); disulphide bonds, A246C (Fierobe et al. (1996), Biochemistry, 35: 8698-8704); and introduction of Pro residues in positions A435 and 5436 (Li et al. (1997) Protein Eng. 10: 1199-1204).

Exemplary fungal glucoamylases may also include Trichoderma reesei glucoamylase and its homologues as disclosed in U.S. Pat. No. 7,413,879 (Danisco US Inc., Genencor Division). Glucoamylases may include, for example, T. reesei glucoamylase, Hypocrea citrina var. americana glucoamylase, H. vinosa glucoamylase, H. gelatinosa glucoamylase, H. orientalis glucoamylase, T. konilangbra glucoamylase, T. harzianum glucoamylase, T. longibrachiatum glucoamylase, T. asperellum glucoamylase, and T. strictipilis glucoamylase.

Other glucoamylases contemplated for use herein include Talaromyces glucoamylases, in particular derived from T. emersonii (WO 99/28448), T. leycettanus (U.S. Pat. No. RE 32,153), T. duponti, or T. thermophilus (U.S. Pat. No. 4,587,215). Contemplated bacterial glucoamylases include glucoamylases from the genus Clostridium, in particular C. thermoamylolyticum (EP 135138) and C. thermohydrosulfuricum (WO 86/01831).

Suitable glucoamylases include the glucoamylases derived from Aspergillus oryzae, such as a glucoamylase having about 50%, about 55%, about 60%, about 65%, about 70%, about 75%, about 80%, about 85%, or even about 90% identity to the amino acid sequence disclosed in WO 00/04136. Suitable glucoamylases may also include the glucoamylases derived from T. reesei, such as a glucoamylase having about 50%, about 55%, about 60%, about 65%, about 70%, about 75%, about 80%, about 85%, or even about 90% identity to the amino acid sequence disclosed in WO 08/045,489 (Danisco US Inc., Genencor Division). T. reesei glucoamylase variants with altered properties, such as those disclosed in WO 08/045,489 and US 2009/0275080, filed Nov. 20, 2008 (Danisco US Inc., Genencor Division), may be particularly useful.

Also suitable are commercial glucoamylases, such as Spirizyme® Fuel, Spirizyme® Plus, and Spirizyme® Ultra (Novozymes A/S, Denmark), G-ZYME® 480, G-ZYME® 480 Ethanol, GC 147, DISTILLASE®, and FERMENZYME® (Danisco US Inc., Genencor Division). Glucoamylases may be added in an amount of about 0.02-2.0 GAU/g ds or about 0.1-1.0 GAU/g ds, e.g., about 0.2 GAU/g ds.

### 3.3. Cellulases

Cellulases are capable of hydrolyzing cellulose, which may provide additional source of glucose for fermentation. In addition, the breakdown of cellulose may release some starch molecules that are bound to or associated closely with some portion of the cellulosic material, or entrapped by the cellulosic material.

Any of a variety of cellulases may be used in conjunction with the saccharification processes and methods provided herein. As defined above cellulases herein encompass a number of different enzyme activities including exo- and endo-glucanases, beta glucosidases, hemi-cellulases, xylanases, and others.

Common names for some cellulases (EC 3.2.1.4) include Avicelase, beta-1,4-endoglucan hydrolase, beta-1,4-glucanase. carboxymethyl cellulase (CMCase), celludextrinase, endo-1,4-beta-D-glucanase, endo-1,4-beta-D-glucanohydrolase, endo-1,4-beta-glucanase, and endoglucanase. These enzymes catalyze endohydrolysis of (1,4)-beta-D-glucosidic linkages in cellulose, lignin and cereal beta-D-glucans.

EC 3.2.1.21 beta-glucosidases include amygdalase, beta-D-glucoside glucohydrolase, cellobiase, and gentobiase, which are responsible for hydrolysis of terminal, non-reducing beta-D-glucosyl residues with the resultant release of beta-D-glucose.

Cellulose 1,4-beta-cellobiosidases (EC 3.2.1.91) include 1,4-beta-cellobiohydrolase, 1,4-beta-D-glucan cellobiohydrolase, exo-1,4-beta-D-glucanase, exocellobiohydrolase, and exoglucanase. Such enzymes are able to catalyze hydrolysis of 1,4-beta-D-glucosidic linkages in cellulose and cellotetraose, releasing cellobiose from the non-reducing ends of the chains.

Some examples of commercial cellulase preparations which are suitable for use herein include the ACCELLERASE 1000 and ACCELLERASE 1500 (Danisco US Inc., Genencor Division) complexes used in the Examples herein. Other commercially-available cellulases contemplated for use herein include OPTIMASH formulations (Danisco US Inc., Genencor Division), BIOCELLULASE TRI, and/or BIOCELLULASE A (Quest Intl. (Sarasota, Fla.)), CELLUCLAST 1.5L (Novo Nordisk, (Danbury, Conn.)), CELLULASE TAP10 and/or CELLULASE AP30K (Amano Enzyme (Troy, Va.)), CELLULASE TRL (Solvay Enzymes (Elkhart, Ind.)), ECONASE CE (Alko-EDC (New York, N.Y.)), MULTIFECT CL, MULTIFECT GC (Danisco US Inc (Genencor Division)), and ULTRA-LOW MICROBIAL (ULM) (Iogen, (Ottawa, Canada)).

Cellulases suitable for use herein can also be made by and isolated from microorganisms including species of the genera Trichoderma, Humicola, Aspergillus, Penicillium, Rhizopus, and Sclerotium for example. Many cellulases can be produced in liquid and/or solid state media and methods for the production and/or preparation of active fractions are abundant in the scientific literature.

### 3.4. Pectinases

Pectinases, or pectic enzymes include several different enzymes, for example pectolyase, pectozyme, pectinesterase, and polygalacturonase. Protopectinases can also be considered as pectinases for purposes herein. EC classes that include pectinases are at least EC 3.1.1.11 (pectin methyl esterase), 3.2.1.15 (polygalacturonase), 3.2.1.67 (exopolygalacturonase), 3.2.1.82 (exo-poly-α-galacturonosidase), 4.2.2.2 (pectic lyase), 4.2.2.9 (pectate disaccharide-lyase), 4.2.2.6 (oligogalacturonide lyase), and 4.2.2.10 (pectin lyase). Any of the foregoing alone or in any combination thereof may be used in accordance with the improved saccharification processes provided herein.

Commercial sources of pectinase enzymes include PANZYM (C.H. Boehringer Sohn (Ingelheim, West Germany)), ULTRAZYME (Ciba-Geigy, A.G. (Basel, Switzerland)), PECTOLASE (Grinsteelvaeket (Aarthus, Denmark)), SCLASE (Kikkoman Shoyu, Co. (Tokyo, Japan)), PECTINEX (Schweizerische Ferment, A.G. (Basel, Switzerland)), RAPIDASE (Societe Rapidase, S.A. (Seclin, France)), KLERZYME (Clarizyme Wallerstein, Co. (Des Plaines, USA)), PECTINOL/ROHAMENT (Rohm, GmbH (Darmstadt, West Germany)), and PECTINASE (Biocon Pvt Ltd (Bangalore, India))

### 3.5. Phytases

Phytases are useful for the present disclosure as they are capable of hydrolyzing phytic acid under the defined conditions of the incubation and liquefaction steps. In some embodiments, the phytase is capable of liberating at least one inorganic phosphate from an inositol hexaphosphate (phytic acid). Phytases can be grouped according to their preference for a specific position of the phosphate ester group on the phytate molecule at which hydrolysis is initiated (e.g., as 3-phytases (EC 3.1.3.8) or as 6-phytases (EC 3.1.3.26)). A typical example of phytase is myo-inositol-hexakiphosphate-3-phosphohydrolase.

Phytases can be obtained from microorganisms such as fungal and/or bacterial organisms. Some of these microorganisms include e.g. Aspergillus (e.g., A. niger, A. terreus, A. ficum, and A. fumigatus), Myceliophthora (M. thermophila), Talaromyces (T. thermophilus), Trichoderma spp (T. reesei), and Thermomyces (WO 99/49740). Phytases are also available from Penicillium species, e.g., P. hordei (ATCC No. 22053), P. piceum (ATCC No. 10519), or P. brevi-compactum (ATCC No. 48944). See, e.g., U.S. Pat. No. 6,475,762. In addition, phytases are available from Bacillus (e.g., B. subtilis, Pseudomonas, Peniophora, E. coli, Citrobacter, Enterobacter, and Buttiauxella (see WO2006/043178)).

Commercial phytases are available such as NATUPHOS (BASF), RONOZYME P (Novozymes A/S), PHZYME (Danisco A/S, Diversa), and FINASE (AB Enzymes). The Maxalig™ ONE (Danisco US Inc., Genencor Division) blend contains a thermostable phytase that is capable of efficiently reducing viscosity of the liquefact and breaking down phytic acid. The method for determining microbial phytase activity and the definition of a phytase unit has been published by Engelen et al. (1994) J. of AOAC Int., 77: 760-764. The phytase may be a wild-type phytase, a variant, or a fragment thereof.

In one embodiment, the phytase is one derived from the bacterium Buttiauxiella spp. The Buttiauxiella spp. includes B. agrestis, B. brennerae, B. ferragutiase, B. gaviniae, B. izardii, B. noackiae, and B. warmboldiae. Strains of Buttiauxella species are available from DSMZ, the German National Resource Center for Biological Material (Inhoffenstrabe 7B, 38124 Braunschweig, Germany). Buttiauxella sp. strain P1-29 deposited under accession number NCIMB 41248 is an example of a particularly useful strain from which a phytase may be obtained and used according to the present disclosure. In some embodiments, the phytase is BP-wild-type, a variant thereof (such as BP-11) disclosed in WO 06/043178, or a variant as disclosed in US 2008/0220498. For example, a BP-wild-type and variants thereof are disclosed in Table 1 of WO 06/043178.

### 3.6. Other Enzymes

In another aspect, the use of a beta-amylase is also contemplated. Beta-amylases (EC 3.2.1.2) are exo-acting maltogenic amylases, which catalyze the hydrolysis of 1,4-α-glucosidic linkages in amylose, amylopectin, and related glucose polymers, thereby releasing maltose. Beta-amylases have been isolated from various plants and microorganisms (Fogarty et al., Progress in Industrial Microbiology, Vol. 15, pp. 112-115, 1979). These beta-amylases are characterized by having optimum temperatures in the range from about 40° C. to about 65° C., and optimum pH in the range from about 4.5 to about 7.0. Contemplated β-amylases include, but are not limited to, beta-amylases from barley Spezyme® BBA 1500, Spezyme® DBA, Optimalt™ ME, Optimalt™ BBA (Danisco US Inc., Genencor Division); and Novozym™ WBA (Novozymes A/S).

Another enzyme that can optionally be added is a debranching enzyme, such as an isoamylase (EC 3.2.1.68) or a pullulanase (EC 3.2.1.41). Isoamylase hydrolyses α-1,6-D-glucosidic branch linkages in amylopectin and β-limit dextrins and can be distinguished from pullulanases by the inability of isoamylase to attack pullulan and by the limited action of isoamylase on α-limit dextrins. Debranching enzymes may be added in effective amounts well known to the person skilled in the art.

### EXAMPLES

The following examples are not to be interpreted as limiting, but are exemplary means of using the methods disclosed.

### Example 1

### Ethanol Production in Samples with Added Cellulase

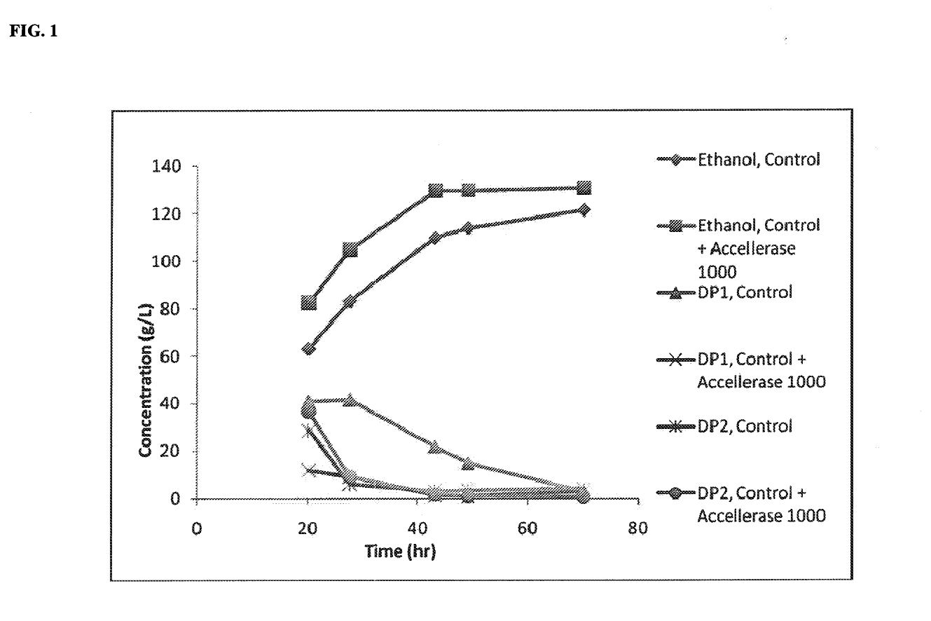

Corn liquefact was obtained from Badger State Ethanol, WI. The dry solid (DS) of the corn liquefact was determined to be 32% DS. Fermentation of corn mash was carried out in duplicate in a 250 ml shake flask containing 100 g of mash. Glucoamylase (G-ZYME 480, Danisco US Inc., Genencor Division) was added at 0.4 GAU/g ds as the control. Cellulase was added at 5.0 kg/MT ds. The cellulase used for these experiments was ACCELLERASE 1000 (Danisco US Inc., Genencor Division), a commercial product containing exo- and endo-glucanase activities, hemicellulase and beta glucosidase. See ACCELLERASE™ 1000 product information sheet, Danisco US Inc., Genencor Division. Yeast (Saccharomyces cerevisiae) was added to liquefact at a concentration of 0.1% w/w to initiate the fermentation. Incubation temperature was 38° C., with shaking at 150 rpm. Samples were withdrawn at specific time intervals and analyzed for ethanol and residual glucose by HPLC method. Ethanol yield was determined for each sample. The results are shown in FIG. 1.

As can be seen, the data show that inclusion of cellulase substantially improved ethanol yield from the fermentations as compared to the control fermentation containing glucoamylase but no cellulase. It can also be seen in FIG. 1 that the concentration of DP2 in the cellulase-treated fermentation fell more quickly that that in the control, showing that the DP2 was utilized more readily with the cellulase addition than without.

### Example 2

### Comparison of Effect of Cellulase Addition on Ethanol Production

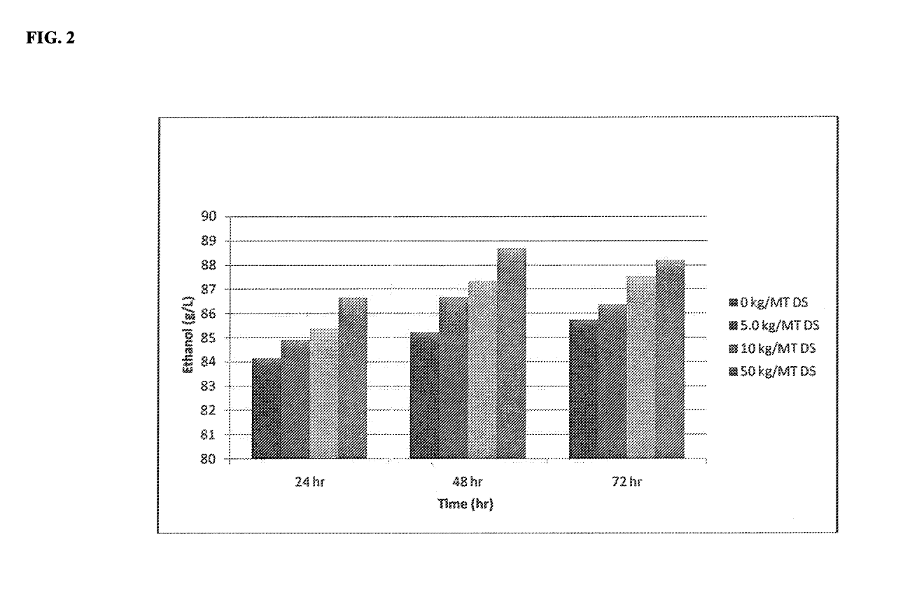

Corn liquefact was prepared by Genencor's Grain Applicants Lab in Beloit, Wis. Ground corns were slurried to obtain 32% DS, and the pH of the slurry was adjusted to pH 5.8. Alpha amylase (SPEZYME® XTRA, Danisco US Inc., Genencor Division) was dosed at 2 AAU/g DS. The slurry was then jet cooked at 107° C. The mash liquefact was subsequently held at 85° C. for 90 minutes with an additional 2.0 AAU/g DS of alpha-amylase. The final DS of the mash was determined to be 23%. Fermentation of corn mash was carried out in duplicate in 250 ml shake flasks containing 100 g of mash. A commercial glucoamylase (G-ZYME 480, Danisco US Inc., Genencor Division) was added at 0.4 GAU/g ds as the control. Cellulase (ACCELLERASE 1500, Danisco US Inc., Genencor Division) was added at 5, 10, and 50 kg/MT ds in the liquefact. This commercially available cellulase product contains exoglucanase, endoglucanase, hemicellulase and beta glucosidase. See ACCELLERASE™ 1500 product information sheet, Danisco US Inc., Genencor Division. Yeast (S. cerevisiae) was added to liquefact at a concentration of 0.1% w/w. Fermentation was conducted at 38° C., with shaking at 150 rpm for 72 hours. Samples were withdrawn at specified time intervals (24, 48, and 72 h) and analyzed for ethanol and residual glucose by HPLC method. Ethanol yields were determined for each sample. The results are shown in FIG. 2. As can be seen, ethanol yields increased as a function of the amount of added cellulase.

### Example 3

### Effect of Cellulase on Ethanol Yield of Corn Mash Fermentation at 33% DS

Corn mash (33% DS) was obtained from an ethanol plant (Corn Plus, MN). Fermentation of the corn mash was carried out in duplicate in 250 ml shake flasks containing 100 g of mash with 600 ppm urea. A commercial glucoamylase (G-ZYME 480, Danisco US Inc., Genencor Division) was added at 0.4 GAU/g ds as the control. Cellulase (ACCELLERASE 1500, Danisco US Inc., Genencor Division) was added at 0.05-0.2% w/w of dry corn. Yeast (S. cerevisiae) was added to liquefact at a concentration of 0.1% w/w. Fermentation was conducted at 32° C., with shaking at 150 rpm. Samples were withdrawn at specified time intervals and analyzed for ethanol and residual glucose by HPLC method. Ethanol yields were determined for each sample.

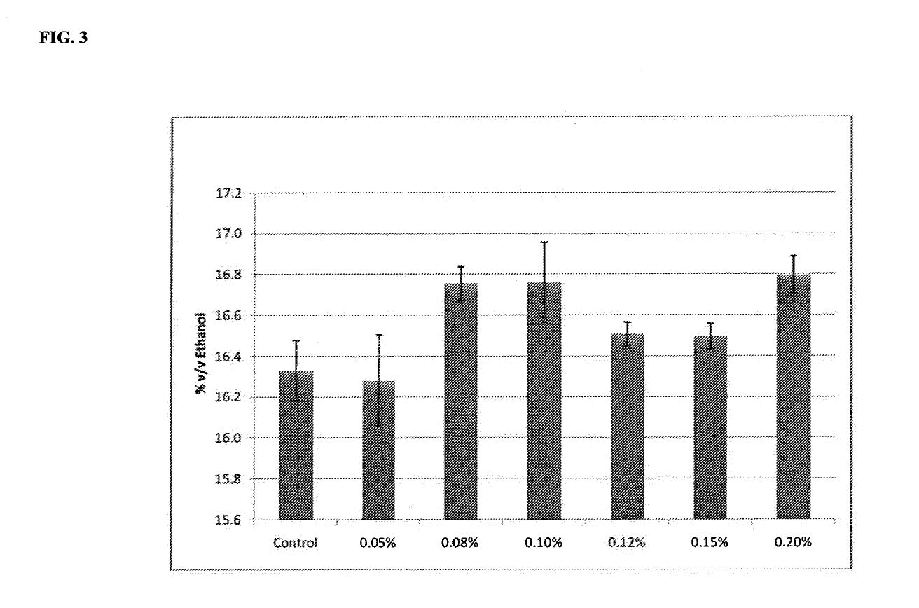

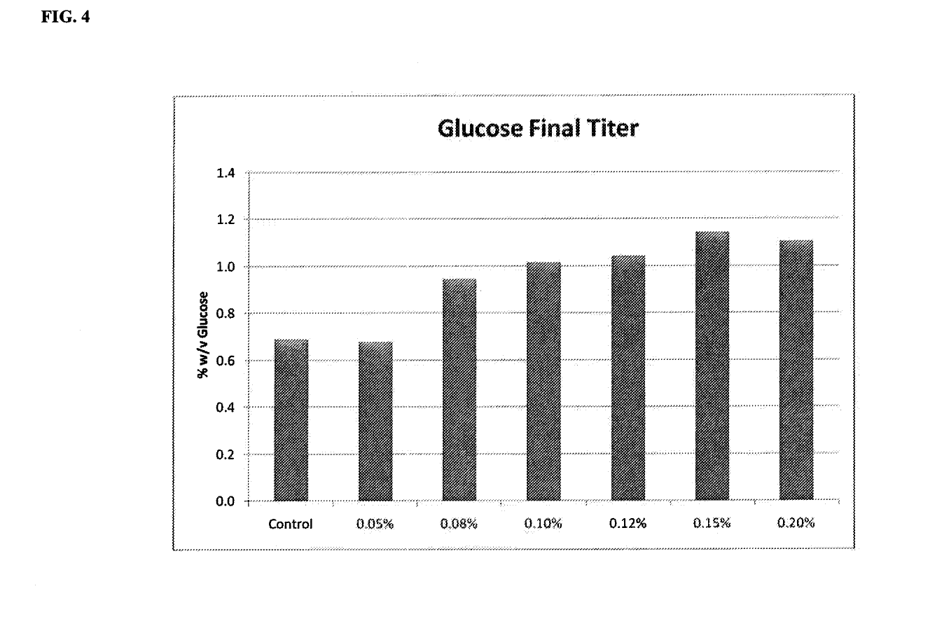

The results are shown in FIGS. 3-4. The results show the final determination made after fermentation for 64 hr. It can be seen that cellulase additions greater than 0.05% w/w provided greater ethanol yields compared to the controls. The benefits, if any, of adding less than about 0.08% cellulase to the SSF process were unclear. As can be seen in FIG. 4, cellulase additions greater than 0.05% resulted in an increase in the final glucose titer, suggesting that even greater yield improvements may be attainable with altered (e.g. longer) fermentation conditions.

While various embodiments have been shown and described herein, it will be clear to those skilled in the art that such embodiments are provided by way of example only. Numerous variations, changes, and substitutions may occur to those skilled in the art without departing from the disclosure.

What is claimed is:

1. A method of saccharifying a starch-containing substrate to prepare a fermentation feedstock comprising (a) contacting a liquefied starch slurry that contains at least some cellulosic material with a glucoamylase and a cellulase under conditions sufficient for enzyme activity, and (b) allowing time for the enzyme activity to occur, thereby producing a fermentation feedstock.

2. The method of claim 1, wherein the enzyme activity is sufficient to at least: (a) increase concentration of at least one fermentable sugar in the fermentation feedstock; (b) release at least one starch chain bound to or trapped by cellulose; or (c) to hydrolyze some portion of the cellulosic material; wherein (a), (b), or (c) may be measured relative to a control liquefied starch slurry not contacted with cellulase.

3. The method of claim 1, wherein the cellulase comprises one or more of exoglucanase, endoglucanase, hemi-cellulase, beta-glucosidase, or xylanase activities, or any combination thereof.

4. The method of claim 3, wherein the cellulase comprises at least exoglucanase, endoglucanase, hemi-cellulase, or beta-glucosidase activities.

5. The method of claim 1, wherein the cellulase is added at between about 0.05 to about 50 kg/metric ton of dry solids in the liquefied starch slurry.

6. The method of claim 5, wherein the cellulase is added at about 5 kg/metric ton dry solids.

7. The method of claim 1 further comprising contacting the liquefied starch slurry with one or more additional enzymes selected from the group consisting of a debranching enzyme, a pectinase, a beta amylase, and a phytase.

8. The method of claim 1, further comprising fermenting the fermentation feedstock to produce a fermentation product.

9. The method of claim 8, wherein the fermentation product is ethanol.

10. The method of claim 8, wherein yield of the fermentation product is higher by about 0.1 to about 1.0%, compared to the yield without the cellulase.

11. The method of claim 10, wherein yield of the fermentation product is higher by about 0.3 to about 0.5%, compared to the yield without the cellulase.

12. The method of claim 8, wherein the contacting and fermenting steps collectively take about 24 to 72 hours.

13. The method of claim 1, wherein starch is from corn, wheat, barley, sorghum, rye, potatoes, or any combination thereof.

14. The method of claim 13, wherein the starch is from corn.

15. The method of claim 8 further comprising contacting the liquefied starch slurry with one or more enzymes selected from the group consisting of a debranching enzyme, a pectinase, a beta amylase, and a phytase.

16. The method of claim 8, wherein the fermenting is done simultaneously with the saccharifying.

17. A composition comprising a liquefied starch slurry, a glucoamylase, and a cellulase.

18. The composition of claim 17, wherein the cellulase is present at between about 0.05 to about 50 kg/metric ton of dry solids.

19. The composition of claim 17, wherein starch is from corn, wheat, barley, sorghum, rye, potatoes, or any combination thereof.

20. The composition of claim 19, wherein the starch is from corn.

21. The composition of claim 17 further comprising one or more additional enzymes selected from the group consisting of a debranching enzyme, a pectinase, a beta amylase, and a phytase.
